(19) United States
(12) Patent Application Publication
(10) Pub. No.: US 2015/0296779 A1
(43) Pub. Date: Oct. 22, 2015

(54) SUBSTITUTED DIHYDROPYRIDINES FOR SOMATIC EMBRYOGENESIS IN PLANTS

(71) Applicant: STICHTING DIENST LANDBOUWKUNDIG ONDERZOEK, Wageningen (NL)

(72) Inventors: Martijn FIERS, Bennekom (NL); Kimberly BOUTILIER, De Meern (NL)

(73) Assignee: STICHTING DIENST LANDBOUWKUNDIG ONDERZOEK, Wageningen (NL)

(21) Appl. No.: 14/647,667

(22) PCT Filed: Nov. 28, 2012
(86) PCT No.: PCT/IB2012/056789

§ 371 Date: May 27, 2015

(51) Int. Cl. A01N 43/42; A01N 43/40; C07D 401/04; A01N 43/38

(52) U.S. Cl. CPC A01N 43/42; A01N 43/38; A01N 43/40; C07D 401/04

(57) ABSTRACT

Cyclopentyl 2,7,7-trimethyl-5-oxo-4-(4-pyridinyl)-1,4,5,6,7,8-hexahydro-3-quinolinecarboxylate and similar compounds are potentiators of auxin-induced somatic embryogenesis in plants. In particular, the inventors have discovered certain of these compounds induce somatic embryogenesis in Arabidopsis in the presence of 2,4-D. Also tested is BAY K 8644. Methods of inducing somatic embryogenesis comprise exposing selected plant tissues, e.g. seed embryos, to auxins, e.g. 2.4-D and the compounds.

This invention relates to somatic embryogenesis (SE) in plants which is the formation of plant embryos from vegetative or somatic plant cells, including somatic embryos made from seed embryos. The invention concerns compounds and compositions which affect plant cells' ability form somatic embryos.

Plant regeneration and clonal propagation are important techniques in agricultural and horticultural sectors, where they are used to facilitate the breeding process, to propagate parental lines for hybrid seed production, and to propagate highly heterozygous or open pollinated varieties that are sold as plantlets.

One technique used to clonally propagate plant material is SE in which embryo-like structures can develop into fertile plants in a way analogous to zygotic embryos that develop in the seed.

SE has several advantages compared to other in vitro clonal propagation systems, such as the possibility to obtain a high yield of plants in a short time, the possibility to scale-up in liquid suspension cultures and synthetic seed technologies.

In the model system of Arabidopsis thaliana, immature seedlings can be induced in a highly efficient manner to form somatic embryos with the aid of the synthetic auxin 2,4 dichlorophenoxyacetic acid (2,4-D). This is in contrast to zygotic embryos from dry seed, which have a reduced competence to form somatic embryos.

Considering plants generally, a complicating factor is a large variability in SE efficiency between different cultivars and species. Transferring SE protocols from model genotypes to other plant species poses problems and germplasm recalcitrance of commercial cultivars is a major continuing problem. In order to be able to induce SE in recalcitrant cultivars an empirical approach, based on existing tissue culture protocols, hitherto, needs to be used to identify optimal conditions and inducer treatments for each individual genotype. These processes though are hugely time consuming and may not result in an efficient somatic embryogenesis protocol.

A genetic modification (GMO) approach, in which regeneration-promoting genes are (conditionally) over-expressed may be used, but this approach requires a transformable genotype.

The scientific literature comprises many publications of the various methods of somatic embryogenesis, each method involving culturing explants of tissue, cells or callus in a specifically elucidated growth medium comprising plant growth regulators (PGRs). Gaj M. D. (2004) et al. Plant Growth Regulation 43: 27-47 is a review article which summarises the general characteristics of SE and considers the various factors which are crucial for SE induction; including the growth medium compositions and PGRs. Of the PGRs used in SE induction media, 2,4-D is the more frequently reported example which induces an embryogenic response, whether alone or in combination with other PGRs, across a wide range of in vitro systems and plant tissues. Whilst 2,4-D is almost routinely reported as being used to induce SE, other auxins and cytokines, whether alone or in combination in growth media, have been reported to induce embryogenesis.

The published patent literature in the field of somatic embryogenesis to date is characteristically reflective of the science, whereby a method of embryogenesis is developed empirically for a particular plant species or cultivar, usually of an ornamental, horticultural or agricultural plant; but also including conifer trees.

Thus there is a need in the context of plant breeding and plant improvement to enhance or develop generically applicable methods for efficiently enhancing SE in a cost-effective manner.

The inventors have discovered that cyclopentyl 2,7,7-trimethyl-5-oxo-4-(4-pyridinyl)-1,4,5,6,7,8-hexahydro-3-quinolinecarboxylate and similar compounds are excellent potentiators of 2,4-D induced embryogenesis in Arabidopsis. In particular, the inventors have discovered certain of these compounds induce SE in Arabidopsis in the presence of 2,4-D, some strongly so. The inventors also tested compound known as BAY K 8644 (a presumed calcium channel activator) which is an analogue of the cyclopentyl 2,7,7-trimethyl-5-oxo-4-(4-pyridinyl)-1,4,5,6,7,8-hexahydro-3-quinolinecarboxylate.

In accordance with the present invention there is provided a method of potentiating auxin-induced somatic embryogenesis or organogenesis in a plant, comprising exposing plant cells, plant tissue, plant part, seedling or plant embryo to an auxin and to one or more compounds of the structure:

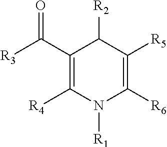

wherein:
   R₁ is hydrogen or a C₁ to C₆ hydrocarbyl group, preferably a C₁ to C₆ alkyl group;
   R₂ is an optionally substituted phenyl or pyridinyl ring;
   R₆ is hydrogen, hydroxyl or a C₁ to C₆ hydrocarbyl group, preferably a C₁ to C₆ alkyl group;
   R₅ is —CN or L₁R₇, where L₁ is a linker of the formula C(O)O or CO(O) and R₇ is a C₁ to C₆ alkyl group, and either
   i) R₃ and R₄ together form a 5 or 6 membered cyclic ring, or
   ii) R₃ is R₈ or OR₈, where R₈ is a C₁ to C₆ alkyl group and R₄ is a C₁ to C₆ alkyl group;
and then culturing the cells, tissue, part or embryo.
Protocols and techniques for culturing plant cells, tissues, parts or embryos will be well known to a person of average skill in the art for a wide range of plant species; see for example, the textbook “Plant Cell Culture” (2010) Michael R Davey and Paul Anthony (Wiley-Blackwell). Also, for example, the laboratory methods book “Plant Cell Culture Protocols” (2006) 2nd edition, Victor M Loyola-Vargas and Felipe Vazquez-Flota (Humana Press).
Preferably, the compounds have the structure:

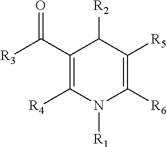

wherein:
   R₁ is hydrogen or a C₁ to C₆ hydrocarbyl group, preferably a C₁ to C₆ alkyl group;
   R₂ is an optionally substituted phenyl or pyridinyl ring;
   R₆ is hydrogen or a C₁ to C₆ hydrocarbyl group, preferably a C₁ to C₆ alkyl group;
   R₅ is of the formula L₁R₇, where L₁ is a linker of the formula C(O)O or CO(O) and R₇ is a C₁ to C₆ alkyl group, and either
   i) R₃ and R₄ together form a 5 or 6 membered cyclic ring, or
   ii) R₃ is R₈ or OR₈, where R₈ is a C₁ to C₆ alkyl group, and R₄ is a C₁ to C₆ alkyl group;

Potentiating the SE activity of auxin is defined as increasing or decreasing the somatic embryo-inducing activity of auxin when used alone without the one or more compounds of the invention. Compounds in accordance with the invention which decrease the SE inducing activity of auxin are equally useful as compounds which increase SE activity as they may be used in studies to elucidate genetic and biochemical basis of SE in plants and to prevent over-proliferation of tissues in culture so that differentiation and subsequent plantlet growth may occur.

Advantageously, the ability of the present invention using certain of the defined compounds to chemically induce SE eliminates the need to create and market transgenic plants, allowing rapid and cost-effective innovation.

Advantageously, the methods of the invention rapidly induce/enhance SE in an inexpensive, non-GMO manner.

Also advantageously, in relation to industrial application, compounds for use in accordance with the invention can be directly tested and implemented in any SE protocol, including existing protocols, without having to perform additional studies to define specificity or mode of action of the compounds.

Advantageously, the invention offers plant breeders the capability of inducing or enhancing SE in a range of different crops.

The compounds of the invention may also be used in potentiating (i.e. increasing or decreasing) different types of auxin-mediated organogenesis, preferably to enhance plantlet formation.

In this specification, the term “hydrocarbyl” refers to substituents consisting of hydrogen and carbon. Such groups may be saturated or unsaturated. For example, the hydrocarbyl may include one or more double or triple carbon-carbon bonds. The hydrocarbyl may be aliphatic or aromatic. Suitable hydrocarbyl groups include straight chain, branched chain and cyclic (e.g. alicyclic or aromatic) groups.

Unless indicated otherwise, the term “alkyl” includes cyclic, straight and branched chain alkyl groups. References to individual alkyl groups such as “n-propyl” are specific for the straight chain version only and references to individual branched chain alkyl groups such as “isopropyl” are specific for the branched chain version only. For example, C₁ to C₄ alkyl includes methyl, ethyl, n-propyl, i-propyl, n-butyl, t-butyl and sec-butyl. Suitable cyclic alkyls include saturated and unsaturated cyclic alkyls. Examples include cyclohexyl and cyclopentyl.

Where R₂ is a substituted phenyl or pyridinyl ring, it may be a mono-substituted phenyl or pyridinyl ring. Where R₂ is substituted, the substituent is preferably a hydrocarbyl group, more preferably a C₁ to C₆ hydrocarbyl group, such as a C₁ to C₆ alkyl group. This alkyl group is preferably non-cyclic. Preferably, the alkyl is a methyl or ethyl group.

In another group of preferred compounds, R₂ is not a group of the formula below:

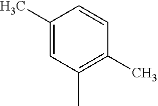

R₁ is preferably a hydrogen or a methyl group.

R₇ may be a straight chain, branched or cyclic (C₃ to C₆) alkyl group. Preferably, R₇ is selected from methyl, ethyl, cyclopentyl or cyclohexyl group.

Preferably, L₁ is a linker of the formula C(O)O.

Where R₆ is a C₁ to C₆ hydrocarbyl, it is preferably a straight chain or branched C₁ to C₆ hydrocarbyl, such as a C₁ to C₆ alkyl. Preferred alkyls include methyl and ethyl.

In another group of preferred compounds, R₃ is R₈ or OR₈, where R₈ is a methyl or ethyl group, and R₄ is a methyl group.

In another group of preferred compounds, R₃ and R₄ together form a 5 or 6 membered cyclic ring. This ring may optionally substituted with at least one C₁ to C₆ alkyl group, preferably a methyl group. For example, the compounds may be of the general formula:

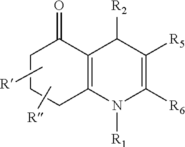

where R′ and R″ are independently selected from hydrogen and C₁ to C₆ alkyl.

In another group, preferred compounds have the formula:

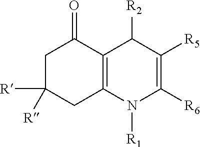

where R′ and R″ are both hydrogen or both methyl.

In another group of preferred compounds, R₂ is selected from

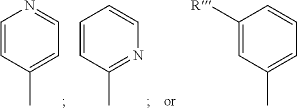

where R′″ is a C₁ to C₆ alkyl group, preferably methyl.

In another group of preferred compounds, R₂ is

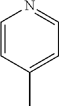

In another group, preferred compounds are selected from:

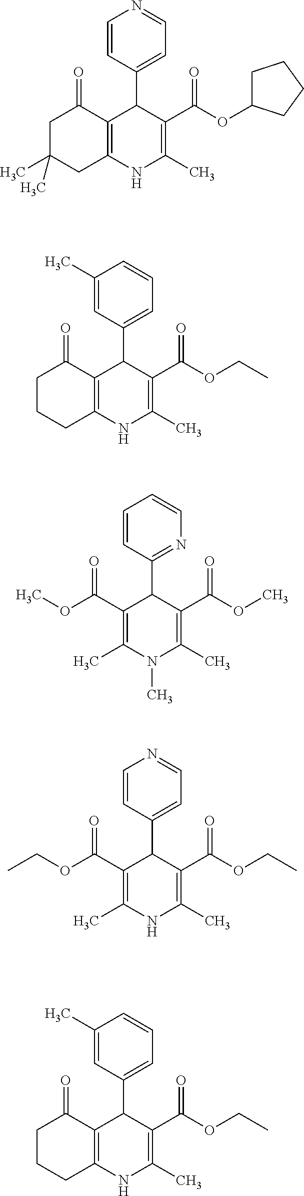

In another group, preferred compounds do not include:

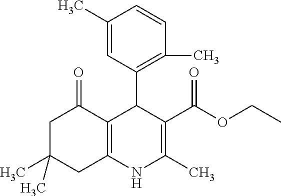 [SMILES: CCOC(=O)C1=C(C)NC2=C(C(=O)CC(C)(C)C2)C1c1cc(C)ccc1C]

A further example is:

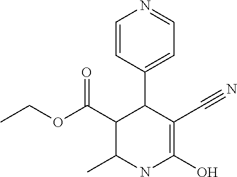 [SMILES: CCOC(=O)C1C(C)NC(O)=C(C#N)C1c1ccncc1]

The above may also be depicted in its keto form:

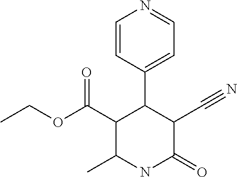 [SMILES: CCOC(=O)C1C(C)NC(=O)C(C#N)C1c1ccncc1]

For the avoidance of doubt, the compound may be used in any suitable form, for example, as a salt or in solvated form. Compounds of the invention may also exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below)

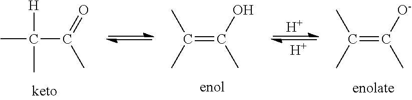

Specific examples of compounds according to the invention are shown in FIG. 1 of the drawings.

For the avoidance of doubt, the compounds of the present invention may be used in salt or solvated form.

In accordance with the method of the invention, the plant cells, tissue, part or embryo may be, or may include, callus. Also, in accordance with the method of the invention, an embryo used as starting material may itself be a somatic embryo. Often a somatic embryo may be used to generate more somatic embryos in a process known as secondary somatic embryogenesis and the method of the invention defined herein includes secondary somatic embryogenesis.

In certain embodiments the plant cells, tissue, part or embryo may be exposed to the auxin and the one or more compounds substantially simultaneously.

In other embodiments the plant cells, tissue, part or embryo may be exposed to the auxin followed by the one or more compounds whether separately, sequentially or simultaneously.

In yet further embodiments, the plant cells, tissue, part or embryo may be exposed to the one or more compounds, whether separately, sequentially or simultaneously, followed by the auxin.

In other embodiments of the invention, there may be a first period of exposure followed by a second period of culturing in the absence of both of the auxin and the one or more compounds.

In any of the embodiments of the method of the invention, the exposing of the plant cells, tissue, part or embryo may take place in a liquid medium. The exposure may also take place at some stage in liquid medium, semi-solid medium or via solid medium, or use of all three in any desired order at any desired time during the conduct of the method of the invention.

Further, the culturing of cells, tissue, part or embryo following any exposure may take place on a solid medium.

In methods of the invention, an optimal window of culture time for the plant cells, tissue, part or embryo (including seeds/seedlings) with the auxin and compound of the invention may be determined in a routine way by a person of skill in the art. In preferred methods, the auxin and compound of the invention (whether used separately, sequentially or simultaneously) are applied within the first three days or culture, more preferably the first two days or first day. In other embodiments, the exposure to compound of the invention (with auxin whether applied separately, sequentially or simultaneously) may be no longer than about two days, preferably no longer than about three days from the start of the culturing process. In preferred embodiments of generating somatic embryos from germinating seeds, the culture time with auxin and compound of the invention no more than about a day, preferably no more than about two days.

The auxin used in accordance with the invention may be selected from one or more of: indole-3-acetic acid (IAA), indole-3-butyric acid (IBA), 4-chloroindole-3-acetic acid (4-Cl-IAA), 2-phenylacetic acid (PAA), 2.4-dichlorophenoxyacetic acid (2,4-D), α-napthalene acetic acid (α-NAA), 2-methoxy-3,6-dichlorobenzoic acid, 4-amino-3,5,6-trichloropicolinic acid. Any auxin, whether natural or synthetic may be used.

In preferred methods according to the invention, the auxin is 2,4 dichlorophenoxyacetic acid (2,4-D).

The invention also provides a method of generating plantlet or plant, comprising producing a somatic embryo as described herein and then regenerating the plantlet or plant from the embryo.

The invention additionally provides a composition for potentiating somatic embryogenesis or organogenesis in plants comprising an auxin and one or more compounds as hereinbefore described.

The invention further includes compounds as hereinbefore described for use in potentiation of somatic embryogenesis or organogenesis in a plant cell, tissue, part or embryo.

The invention also further includes compounds as hereinbefore described for use simultaneously, sequentially or separately with an auxin in the potentiation of somatic embryogenesis or organogenesis in a plant cell, tissue, part or embryo.

Also provided in accordance with the invention is a solid or liquid plant culture medium comprising one or more compounds as hereinbefore described; optionally further comprising an auxin.

The invention also includes a kit for potentiation of somatic embryogenesis or organogenesis in plants, comprising a first container containing a substance which is or comprises one or more compounds as hereinbefore described, and a second container containing a substance which is or comprises an auxin.

In accordance with the invention, the one or more compounds are used at an appropriate concentration, preferably to enhance auxin-mediated SE when they are used at a concentration in the nanomolar to micromolar to millimolar range, preferably in the nanomolar to micromolar range. In particularly preferred embodiments the compounds are used in the range 0.1 to 100 micromolar, more preferably 1 to 50 micromolar.

Preferably, the one or more compounds used in the method of the invention increase the level of SE compared to the auxin alone. A comparative experiment and therefore comparative measurement is preferably made. A preferred measurement of level of SE is the number or weight of embryos produced from a plant cell, tissue, part, embryo, seedling or callus. The number or weight of embryos may be expressed in relation to the experimental material of cells, tissues, parts, embryos, seedlings or callus, i.e. a preferred measure is percentage of seedlings with embryo(s). In preferred embodiments seeking increased level of SE compared to auxin alone, the level of SE may be expressed as a ratio of embryos as measured per unit of experimental material, i.e. number or weight of embryos with one or more compounds of the invention and auxin: number or weight of embryos with auxin alone.

Somatic embryos are readily recognised by a person of average skill in the art. There are distinct morphological characteristics, for example a more mature somatic embryo is bipolar, usually lacking trichomes, is not connected to the underlying vascular tissue and is easily removed from the explant. Younger somatic embryos may be globular in shape, and as above do not contain trichomes and are not connected to the underlying vascular tissue of the explant. Also, there are gene expression markers measurable by qPCR. Such markers show an at least two-fold increase in plant tissue treated with an auxin plus compounds of the invention compared to a control plant tissue treated with just the auxin.

Markers for determining SE may include one or more of the genes described in publicly available embryo transcriptome data sets, including, but not limited to those described at Genevestigator (https://www.genevestigator.com/qv/) or The Bio-Array Resource for Plant Biology (http://bar.utoronto.ca/welcome.htm). Specific examples of such genes include LEAFY COTYLEDON1, LEAFY COTYLEDON2, FUSCA3 and WUSCHEL-RELATED HOMEOBOX2

In all aspects, the invention is applicable to a wide range of plant species, including trees, crop plants, horticultural varieties and ornamentals; including cycads, conifers, angiosperm monocots or dicots. Without purporting to be a comprehensive or exhaustive list of plants susceptible to the compounds and methods of the invention, the following are a list of genera of such plants: e,g, Abies, Pinus, Picea, Tsuga, Pseudotsuga, Thuja, Juniperus, Larix, Taxus and Sequoia. Other plants include, but are not limited to, the genera Elaeis, Phoenix, Eucalyptus, Quercus, Vitis, Malus, Triticum, Oryza, Glycine, Avena, Brassica, Saccharum, Hordeum, Fagopyrum, Gossypium, Beta, Arachis, Humulus, Iopomea, Musa, Manihot, Coffea, Camellia, Rosa, Coca, Canabis, Papaver, Carica, Cocos, Daucus, Medicago, Zea, Solanum Theobroma, Abies, Acer, Alnus, Arbutus, Asimina, Betula, Carpinus, Carya, Castanea, Celtis, Cercis, Chamaecyparis, Cornus, Cryptomeria, Eucalyptus, Fagus, Fraxinus, Gleditsia, Gymnocladus, Hamamelis, Juglans, Juniperus, Larix, Liriodendron, Magnolia, Malus, Morus, Nyssa, Ostrya, Picea, Pinus, Platanus, Populus, Prunus, Pseudotsuga, Pielea, Quercus, Rhamnus, Rhus, Salix, Sambucus, Sassafras, Sequoia, Sorbus, Taxus, Thuja, Tilia, Tsuga, Ulmus, and Viburnum.

In all aspects, the invention is applicable to a wide range of plant species, including trees, crop plants, horticultural varieties and ornamentals.

### BRIEF DESCRIPTION OF THE DRAWINGS

Embodiments of the invention are further described hereinafter with reference to the accompanying drawings, in which:

FIG. 1 shows the results of an experiment testing cyclopentyl 2,7,7-trimethyl-5-oxo-4-(4-pyridinyl)-1,4,5,6,7,8-hexahydro-3-quinolinecarboxylate (compound “C#5”) and similar compounds and analogues for SE activity.

FIG. 2 shows the results of a dosage response experiment in which compound C#5 is tested on seedlings in containers for SE activity. Also shown are pictures allowing comparison of Arabidopsis seedlings with and without somatic embryos.

FIG. 3 shows the results of an experiment in which increasing concentrations of 2,4-D are used to stimulate embryogenesis in Arabidopsis seedlings with or without 25 μM compound C#5.

FIG. 4 shows the results of a dosage response experiment in which compound C#5 is tested on Arabidopsis seedlings in containers.

### DETAILED DESCRIPTION

### Example 1

### Screening of Compounds for Somatic Embryogenesis Activity

The chemical screen was based on a modified version of an Arabidopsis somatic embryogenesis protocol that uses germinated seeds as explants (see Kobayashi et al., 2010 Kobayashi T, Nagayama Y, Higashi K and Kobayashi M. (2010). Establishment of a tissue culture system for somatic embryogenesis from germinating embryos of Arabidopsis thaliana Plant Biotech. 27: 359-364). In the original protocol, seeds were germinated on solid medium for one day, then the embryos were removed from the seed coat and transferred to solid media containing 4.5 μM 2,4-D to stimulate somatic embryo development. In this example, the protocol is simplified by germinating and culturing the seeds continuously in liquid medium containing 1 μM 2,4-D. Under optimized culture conditions (circa 30 seeds in 30 ml medium) approximately 18% of Col0 seeds form somatic embryos.

The LATCA (Library of AcTive Compounds on Arabidopsis (LATCA) library (see Zhao et al., (2007) Zhao Y, Chow T F, Puckrin R S, Alfred S E, Korir A K, Larive C K, Cutler S R (2007) Chemical genetic interrogation of natural variation uncovers a molecule that is glycoactivated. Nat Chem Biol. 3:716-2 was screened for small molecules that enhance the frequency of somatic embryo induction from germinated seeds. The screens are performed in 96-well microtitre plates. For the primary screen, 2.5 μl of each compound (2.5 mM stock in dimethyl sulfoxide (DMSO)) was added to 250 μl of half strength Murashige and Skoog (MS) medium with micro and micro elements and vitamins (Duchefa) and 1% (w/v) sucrose (pH 5.8, MS10) containing 1 μM 2,4-dichlorophenoxyacetic acid (2,4-D) in 96-well microtitre plates.

Approximately 10 surface-sterilized Columbia (Col-0) seeds were added to each well. The seeds were stratified at 4° C. in the dark for two days and then grown in on a rotary shaker (100 rpm/min) on a 16 h/8 h day/night cycle at 25° C. The number of seedlings that formed somatic embryos was counted two weeks later. Candidate compounds identified in the first screen were reordered (Chembridge or Maybridge), and re-tested in a second screen under the same conditions as the primary screen, but with a larger sample number (one 96-well microtitre plate/compound). Positive compounds were analysed using ChemMine (http://bioweb.ucr.edu/ChemMineV2/), ChemSpider (http://www.chemspider.com/) and the ZINC database (http://zinc.docking.org) for analogues with a putative biological function.

Analogues of compound 5 (cyclopentyl 2,7,7-trimethyl-5-oxo-4-(4-pyridinyl-1,4,5,6,7,8-hexahydro-3-quinolinecarboxylate were obtained).

All subsequent experiments were performed under the conditions described above, but using 100 ml containers (Greiner) containing 30 ml of medium media using approximately 30 seeds/container. All hormones were ordered from Duchefa and Dicamba from Sigma-Aldrich.

The effect of the compounds on seedlings, without 2,4-D, was tested by germinating seedlings on MS10 solidified with 1% agar, and supplemented with 25 μM compound or DMSO.

Using the lower response higher throughput screen (10 seeds per well), twenty seven compounds were identified that associated with somatic embryo development. These compounds were re-tested in a 96-well plate (circa 1000 seeds per compound). The compound cyclopentyl 2,7,7-trimethyl-5-oxo-4-(4-pyridinyl)-1,4,5,6,7,8-hexahydro-3-quinolinecarboxylate was chosen for further analysis.

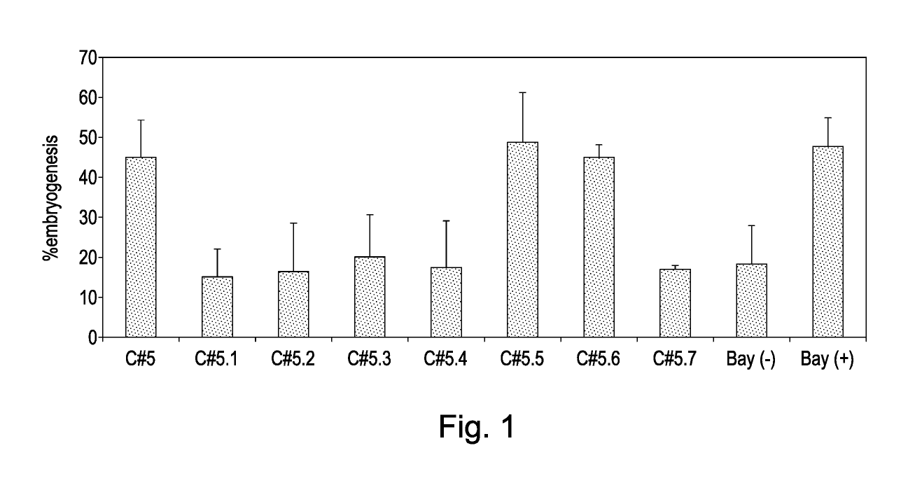

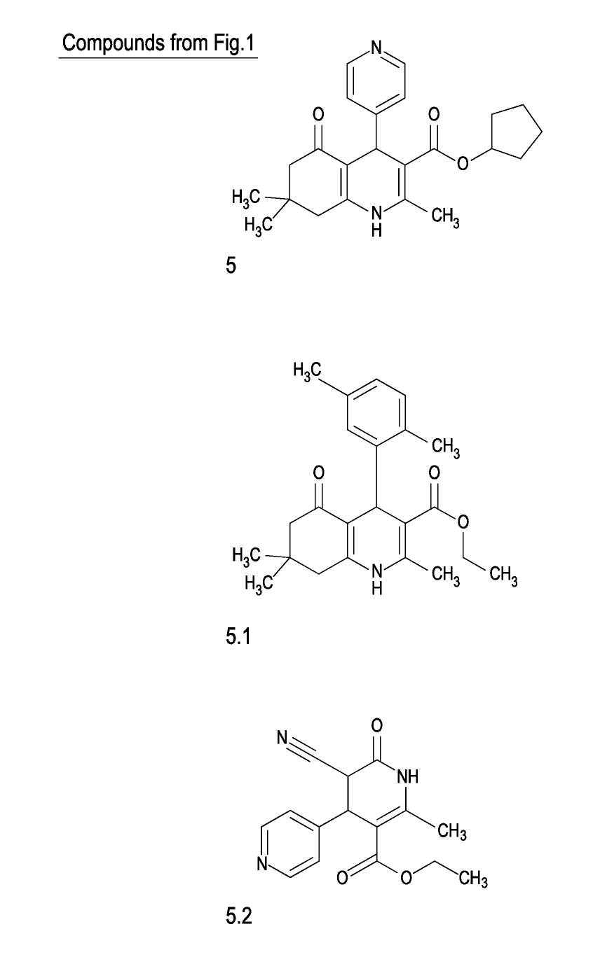

Referring to FIG. 1, this shows the results of testing compounds C#5 and C#5.1-C#5.7 in the presence of 1 μM 2,4-D. The compound Bay is BAY K 8644 (Chemical Name: 1,4-Dihydro-2,6-dimethyl-5-nitro-4-[2-(trifluoromethyl)phenyl]-3-pyridinecarboxylic acid, methyl ester). The (+) and (−) stands for the two separate enantiomers.

Each of the compounds significantly increases the level of SE in Arabidopsis seedlings, as measured by the percentage of seedlings with somatic embryos.

The compounds C#5 and C#5.5 induced a high percentage of seedlings with somatic embryos and C#5 was chosen for further analysis.

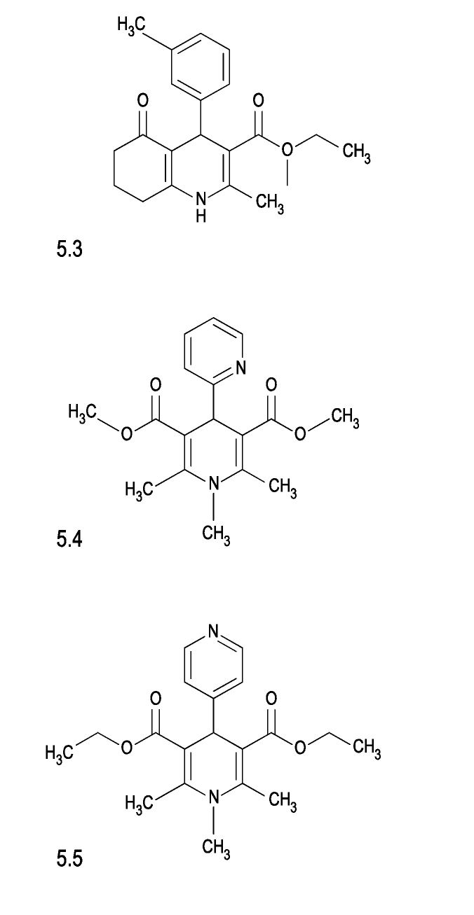

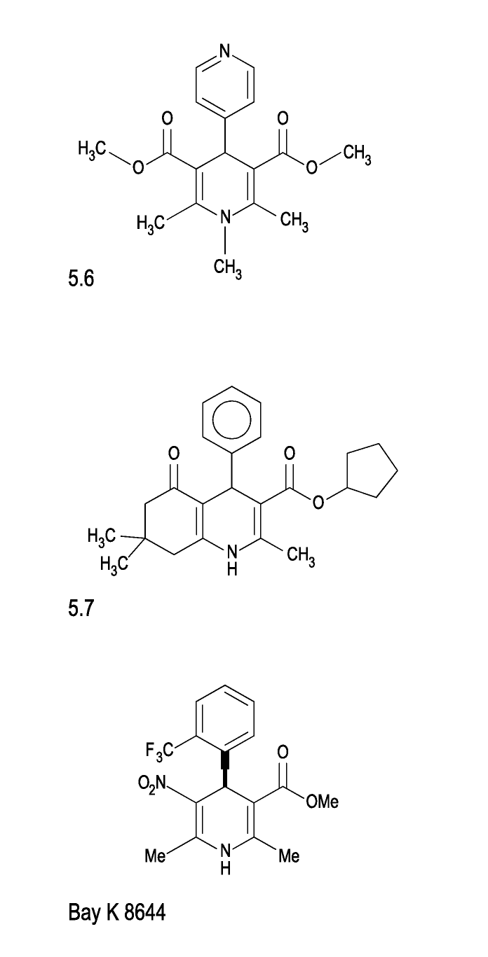

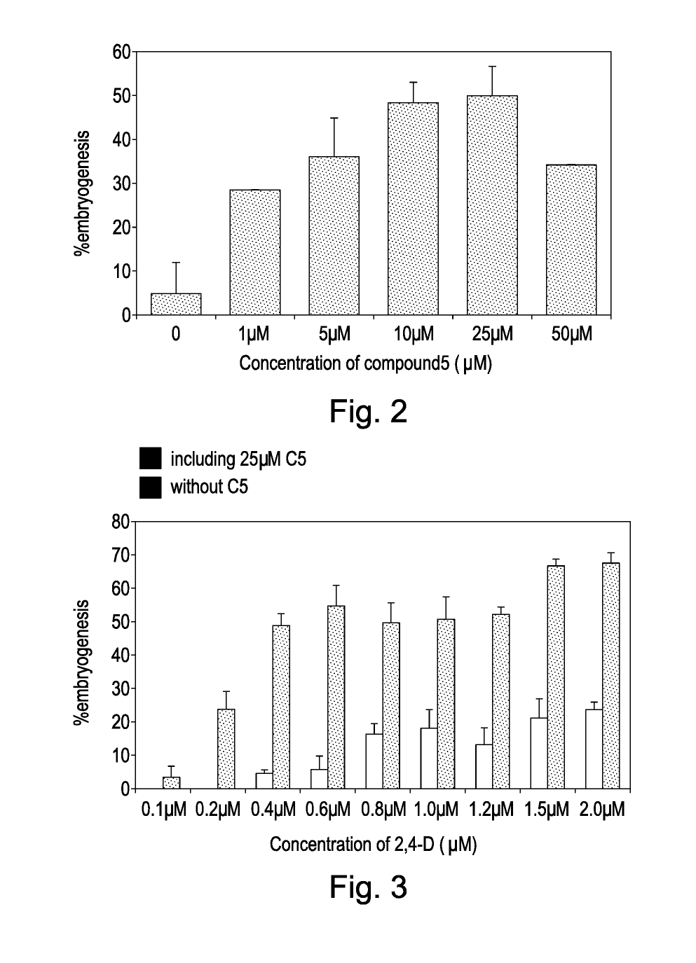

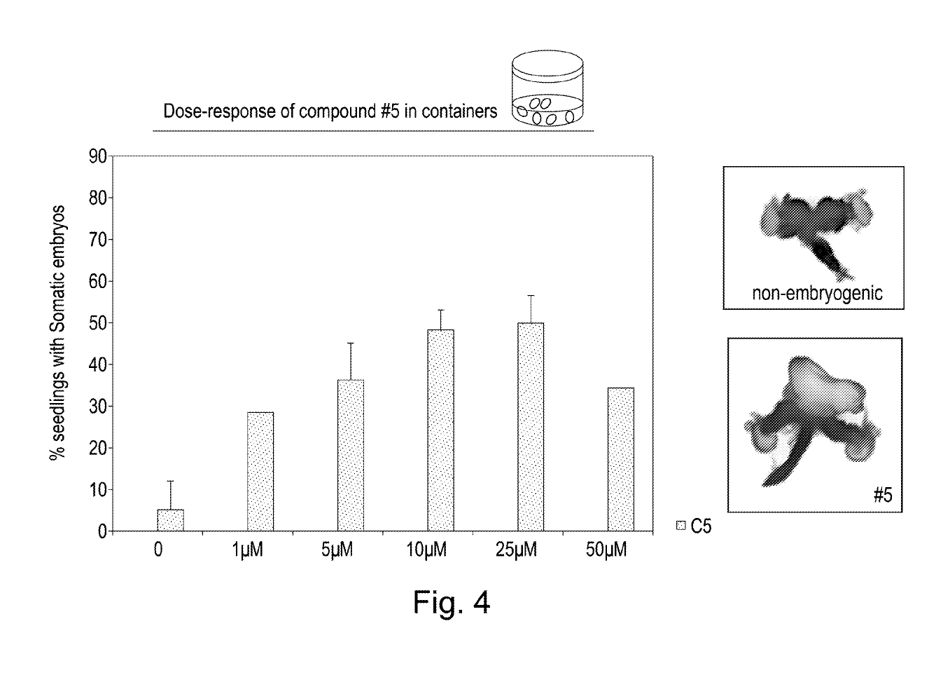

A dose-response analysis was performed under optimized culture conditions (circa 30 seeds in 30 ml medium) and from the results of FIG. 2 and FIG. 4 shows that C#5 is most effective between about 10 and about 50 μM, where it induced somatic embryo development in approximately 50% of seedlings, compared to circa 5% of the seedlings in the control.

Referring to FIG. 3 (Error=SD (n=4), this shows how at a fixed concentration of 25 μM 2,4-D, increasing concentrations of 2,4-D lead to increased levels of SE in Arabidopsis seedlings grown in containers. A threshold concentration of about 0.4 μM 2,4-D is needed to achieve enhanced SE using 25 μM of C#5.

### Example 2

### Cyclopentyl 2,7,7-trimethyl-5-oxo-4-(4-pyridinyl)-1,4,5,6,7,8-hexahydro-3-quinolinecarboxylate (compound C#5) enhances the auxin response

2,4-D is widely used to induce somatic embryogenesis from plant explant. In Arabidopsis immature zygotic embryos occasionally form somatic embryos when grown in basal medium, while germinating embryos or other vegetative tissues are unable to form embryos. The ability of C#5 to enhance somatic embryogenesis was found to be dependent on 2,4-D. Seeds were germinated in medium containing 0 to 2 μM 2,4-D, with or without 25 μM C#5, and then assessed for their ability to form somatic embryos. Compound #5 therefore potentiates the effect of auxin 2,4-D. As shown in FIG. 4, C#5 was not able to induce somatic embryo formation in the absence of 2,4-D, but did enhance somatic embryo formation at all of the tested 2,4-D concentrations, even when no visible somatic embryos were formed in the control (2,4-D only).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of potentiating auxin-induced somatic embryogenesis or organogenesis in a plant, comprising exposing plant cells, plant tissue, plant part, seedling or plant embryo to an auxin and to one or more compounds of the structure: 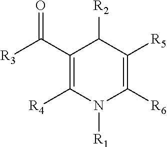
wherein:
R₁ is hydrogen or a C₁ to C₆ hydrocarbyl group, preferably a C₁ to C₆ alkyl group;
R₂ is an optionally substituted phenyl or pyridinyl ring;
R₆ is hydrogen, hydroxyl or a C₁ to C₆ hydrocarbyl group, preferably a C₁ to C₆ alkyl group;
R₅ is —CN or L₁R₇, where L₁ is a linker of the formula C(O)O or CO(O) and R₇ is a C₁ to C₆ alkyl group, and either
i) R₃ and R₄ together form a 5 or 6 membered cyclic ring, or
ii) R₃ is R₈ or OR₈, where R₈ is a C₁ to C₆ alkyl group and R₄ is a C₁ to C₆ alkyl group;
and then culturing the cells, tissue, part or embryo.

2. A method as claimed in claim 1, wherein the compounds have the structure: 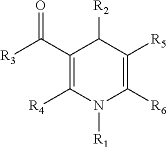
wherein:
R₁ is hydrogen or a C₁ to C₆ hydrocarbyl group, preferably a C₁ to C₆ alkyl group;
R₂ is an optionally substituted phenyl or pyridinyl ring;
R₆ is hydrogen or a C₁ to C₆ hydrocarbyl group, preferably a C₁ to C₆ alkyl group;
R₅ is of the formula L₁R₇, where L₁ is a linker of the formula C(O)O or CO(O) and R₇ is a C₁ to C₆ alkyl group, and either
i) R₃ and R₄ together form a 5 or 6 membered cyclic ring, or
ii) R₃ is R₈ or OR₈, where R₈ is a C₁ to C₆ alkyl group, and R₄ is a C₁ to C₆ alkyl group;

3. A method as claimed in claim 1 or 2, wherein R₂ is an optionally mono-substituted phenyl or pyridinyl ring.

4. A method as claimed in claim 1, 2 or 3, wherein R₂ is optionally substituted with a C₁ to C₆ hydrocarbyl group; preferably a C₁ to C₆ alkyl group.

5. A method as claimed in any of claims 1 to 4, wherein R₂ is not a group of the formula below:

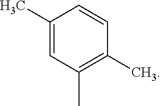

6. A method as claimed in claim 5, wherein R₁ is hydrogen or a methyl group.

7. A method as claimed in any of claims 1 to 6, wherein R₇ is a methyl or ethyl group.

8. A method as claimed in claim 7, wherein L₁ is a linker of the formula C(O)O.

9. A method as claimed in any of claims 1 to 8, wherein R₆ is methyl.

10. A method as claimed in any of claims 1 to 9, wherein R₃ is R₈ or OR₈, where R₈ is a methyl or ethyl group, and R₄ is a methyl group.

11. A method as claimed in any of claims 1 to 9, wherein R₃ and R₄ together form a 5 or 6 membered cyclic ring.

12. A method as claimed in claim 11, wherein the ring is optionally substituted with at least one C₁ to C₆ alkyl group; preferably a methyl group.

13. A method as claimed in claim 1, wherein the compound is of the general formula: 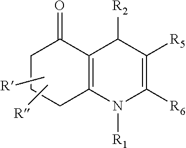
where R′ and R″ are independently selected from hydrogen and C₁ to C₆ alkyl.

14. A method as claimed in claim 13, wherein the compound has the formula: 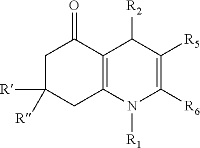
where R′ and R″ are both hydrogen or both methyl.

15. A method as claimed in any of claims 1 to 14, wherein R₂ is selected from 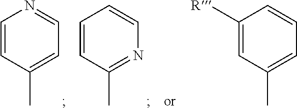
where R′″ is a C₁ to C₆ alkyl group, preferably methyl.

16. A method as claimed in claim 15, wherein R₂ is

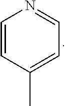

17. A method as claimed in claim 1, wherein the compound is selected from:

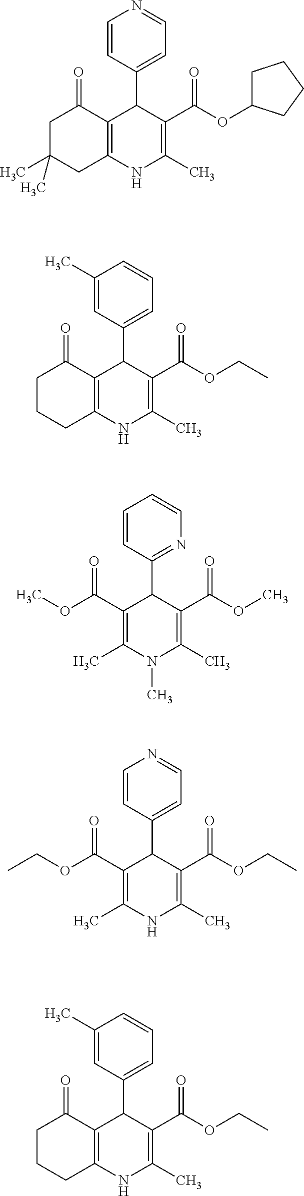

18. A method as claimed in any of claims 1 to 17, wherein the compound is not

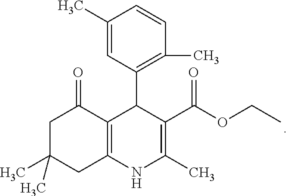 [SMILES: CCOC(=O)C1=C(C)NC2=C(C(=O)CC(C)(C)C2)C1c1cc(C)ccc1C]

19. A method as claimed in any of claims 1 to 18, wherein the plant cells, tissue, part or embryo is, or includes, callus.

20. A method as claimed in any of claims 1 to 18, wherein the plant cells, tissue, part or embryo are exposed to the auxin and the one or more compounds substantially simultaneously.

21. A method as claimed in any of claims 1 to 18, wherein the plant cells, tissue, part or embryo are exposed to the auxin followed by the one or more compounds whether separately, sequentially or simultaneously.

22. A method as claimed in any of claims 1 to 18, wherein the plant cells, tissue, part or embryo are exposed to the one or more compounds, whether separately, sequentially or simultaneously, followed by the auxin.

23. A method as claimed in any of claims 1 to 18, wherein a first period of exposure is followed by a second period of culturing in the absence of both of the auxin and the one or more compounds.

24. A method as claimed in any preceding claim, wherein the exposing of the plant cells, tissue, part or embryo takes place in a liquid medium.

25. A method as claimed in claim 23 or claim 24, wherein culturing of the cells, tissue, part or embryo takes place on a solid medium.

26. A method as claimed in any preceding claim, wherein the auxin is selected from one or more of: indole-3-acetic acid (IAA), indole-3-butyric acid (IBA), 4-chloroindole-3-acetic acid (4-Cl-IAA), 2-phenylacetic acid (PAA), 2.4-dichlorophenoxyacetic acid (2,4-D), α-napthalene acetic acid (α-NAA), 2-methoxy-3,6-dichlorobenzoic acid, 4-amino-3,5,6-trichloropicolinic acid.

27. A method as claimed in claim 26, wherein the auxin is 2,4 dichlorophenoxyacetic acid (2,4-D).

28. A method of generating plantlet or plant, comprising producing a somatic embryo as claimed in any of claims 1 to 27 and then regenerating the plantlet or plant from the embryo.

29. A composition for potentiating somatic embryogenesis or organogenesis in plants comprising an auxin and one or more compounds as defined in any of claims 1 to 18.

30. Compounds of the structure: 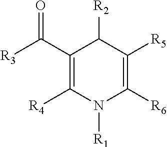
wherein:
R₁ is hydrogen or a C₁ to C₆ hydrocarbyl group, preferably a C₁ to C₆ alkyl group;
R₂ is an optionally substituted phenyl or pyridinyl ring;
R₆ is hydrogen, hydroxyl or a C₁ to C₆ hydrocarbyl group, preferably a C₁ to C₆ alkyl group;
R₅ is —CN or L₁R₇, where L₁ is a linker of the formula C(O)O or CO(O) and R₇ is a C₁ to C₆ alkyl group, and either
i) R₃ and R₄ together form a 5 or 6 membered cyclic ring, or
ii) R₃ is R₈ or OR₈, where R₈ is a C₁ to C₆ alkyl group and R₄ is a C₁ to C₆ alkyl group;
for use in the potentiation of somatic embryogenesis or organogenesis in a plant cell, tissue, part or embryo.

31. Compounds of the structure: 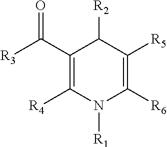
wherein:
R₁ is hydrogen or a C₁ to C₆ hydrocarbyl group, preferably a C₁ to C₆ alkyl group;
R₂ is an optionally substituted phenyl or pyridinyl ring;
R₆ is hydrogen, hydroxyl or a C₁ to C₆ hydrocarbyl group, preferably a C₁ to C₆ alkyl group;
R₅ is —CN or L₁R₇, where L₁ is a linker of the formula C(O)O or CO(O) and R₇ is a C₁ to C₆ alkyl group, and either
i) R₃ and R₄ together form a 5 or 6 membered cyclic ring, or
ii) R₃ is R₈ or OR₈, where R₈ is a C₁ to C₆ alkyl group and R₄ is a C₁ to C₆ alkyl group;
for use simultaneously, sequentially or separately with an auxin in the potentiation of somatic embryogenesis or organogenesis in a plant cell, tissue, part or embryo.

32. Compounds as claimed in claim 30 or claim 31, wherein the compound has a structure as claimed in any of claims 2 to 18.

33. A solid or liquid plant culture medium comprising one or more compounds as set forth in any of claims 1 to 18.

34. A medium as claimed in claim 33, further comprising an auxin.

35. A kit for potentiating somatic embryogenesis or organogenesis in plants, comprising a first container containing a substance which is or comprises one or more compounds as defined in any of claims 1 to 18, and a second container containing a substance which is or comprises an auxin.
